(19) United States
(12) Patent Application Publication
(10) Pub. No.: US 2016/0235935 A1
(43) Pub. Date: Aug. 18, 2016

(54) Endotracheal Tube Securement Device

(71) Applicant: University of Rochester, Rochester, NY (US)

(72) Inventors: M. Zubair Mirza, Rochester, NY (US); Sarah Elizabeth Walsh, Amherst, NY (US)

(73) Assignee: University of Rochester, Rochester, NY (US)

(21) Appl. No.: 15/042,308

(22) Filed: Feb. 12, 2016

Related U.S. Application Data
(60) Provisional application No. 62/116,408, filed on Feb. 14, 2015.

(51) Int. Cl. A61M 16/04; A61M 16/08

(52) U.S. Cl. CPC A61M 16/0493; A61M 16/0816; A61M 16/0463; A61M 16/0497

(57) ABSTRACT

An endotracheal tube securement device is disclosed. The device provides a sanitary and secure way to retain aft endotracheal tube. The device employs bite wedges to prevent a patient from biting the endotracheal tube, while providing an unobtrusive, comfortable and secure means for maintaining the endotracheal tube in place. The device is less visually traumatic than current arrangements. The device is small, with little to no facial contact, providing minimal obstruction of the oral cavity for hygiene and clinical management. The device has a first limb and a second limb joined at a junction that forms a rest for the endotracheal tube. There is a tube retainer attached to the tube rest. A first strut protrudes from the first limb to retain a first bite wedge, and a second strut protrudes from the second limb to retain a second bite wedge. A suction feature may also be provided.

### CROSS REFERENCE TO RELATED PATENT APPLICATIONS

This application claims priority to U.S. patent application Ser. No. 62/116,408 filed Feb. 14, 2015 entitled “Endotracheal Tube Securement Device” by Mirza and Walsh, the entire disclosure of which is incorporated herein by reference.

### BACKGROUND OF THE INVENTION

1. Field of the Invention

The present invention relates generally to medical devices, and more particularly to an endotracheal tube securement device.

2. Description of the Related Art

Securing an endotracheal tube in a patient's mouth is of major importance for the well-being of a patient. During endotracheal intubation, a flexible plastic tube known as an endotracheal tube is inserted within the trachea. This procedure is often done while the patient is under deep sedation or is unconscious. With the help of a laryngoscope to provide proper visualization and to hold the tongue aside, the endotracheal tube is inserted. Most endotracheal tubes are made of a flexible material such as polyvinyl chloride, silicone rubber, or latex rubber. Some specialized endotracheal tubes are made of other materials, or are reinforced with materials such as stainless steel. Endotracheal tubes often have a line of radio opaque material embedded in their wall to make them visible to medical imaging such as with a chest X-ray. All present day endotracheal tubes have an inflatable cuff to seal the trachea and prevent air leakage or aspiration of undesirable materials such as secretions, gastric contents, or the like. The inflatable cuff is usually connected to a pilot balloon with a small tube that runs parallel to the endotracheal tube, and can be inflated and monitored from the pilot balloon. Some endotracheal tubes have a “Murphy Eye” located along the wall of the tube near the main opening of the tube. If the main opening gets obstructed, for example by abutting against the tracheal wall, gas flow can still occur. Most endotracheal tubes range in size from 2 to 11 millimeters in diameter.

Endotracheal tubes are used to deliver oxygen or to administer other gases or anesthetic agents by positive pressure ventilation to a patient in critical or emergency care, or in anesthesia or mechanical ventilation situations. The endotracheal tube is a commonly used medical device that is critical for patient safety. There are numerous variations on the endotracheal tube, each designed to fit a certain medial situation. While there have been great advances in endotracheal tubes over the years, one of the most common techniques to secure the endotracheal tube is with tape. Tape is problematic in many ways. For one, it does not often provide a secure retention technique, or can loosen over time as secretions build up around the tape. Those secretions can also create unhygienic conditions and infection. The secretions around and under the tape can cause irritation and ultimately infection. The tape can also track secretions to other unintended areas, creating irritation and infection elsewhere. There have been various attempts to create clamps to retain the endotracheal tube. These clamps are often large, unsightly, uncomfortable, and do not always prevent a patient from biting down on the endotracheal tube, and create difficulties in sectioning out secretions with a suction catheter due to their size and overall geometry. Without the ability to thoroughly suction out secretions, there exists a real and serious risk for infection and pneumonia, particularly if the secretions track down the endotracheal tube, creep past the balloon and into the lungs. Further, with both tape as well as various clamps, a patient can bite down on the endotracheal tube, damaging or pinching off the tube and creating an emergency situation.

What is therefore needed is a device for positively securing an endotracheal tube that is small, adjustable, less visually traumatic, and provides minimal obstruction to the oral cavity for hygiene and clinical management. What is also needed is a device that has minimal contact with the face, and with no pressure points. What is further needed is a device that prevents a patient from biting the endotracheal tube, and provides a rest for the jaws.

### BRIEF SUMMARY OF THE INVENTION

In accordance with the present invention, there is provided an endotracheal tube securement device comprising a first limb that generally conforms to a patient's face and having a first strap retainer, a second limb that generally conforms to a patient's face and having a second strap retainer, the first limb joined, to the second limb at a junction forming a tube rest for a tube, a tube retainer movably attached to the tube rest, a first strut protruding from the first limb, the first strut retaining a first bite wedge, and a second strut protruding from the second limb, the second strut retaining a second bite wedge.

The foregoing paragraph has been provided by way of introduction, and is not intended to limit the scope of the invention as described by this specification, claims and the attached drawings.

### BRIEF DESCRIPTION OF THE DRAWINGS

The invention will be described by reference to the following drawings, in which like numerals refer to like elements, and in which:

FIG. 1 depicts a perspective view of the endotracheal tube securement device;

FIG. 2 is a perspective view of an alternate embodiment of the endotracheal tube securement device;

FIG. 3 is a front perspective view of the endotracheal tube securement device of FIG. 1;

FIG. 4 is a top plan view of the endotracheal tube securement device of FIG. 1;

FIG. 5 is a front plan view of the endotracheal tube securement device of FIG. 1;

FIG. 6 is a first side plan view of the endotracheal tube securement device of FIG. 1;

FIG. 7 is a second side plan view of the endotracheal tube securement device of FIG. 1;

FIG. 8 is a perspective view of a bite wedge of the present invention; and

FIG. 9 depicts the endotracheal tube securement device in use.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, claims and drawings attached hereto.

### DESCRIPTION OF THE PREFERRED EMBODIMENTS

An Endotracheal Tube Securement Device is described herein. The device represents a significant improvement over the common technique of using tape or clamps for retention of an endotracheal tube.

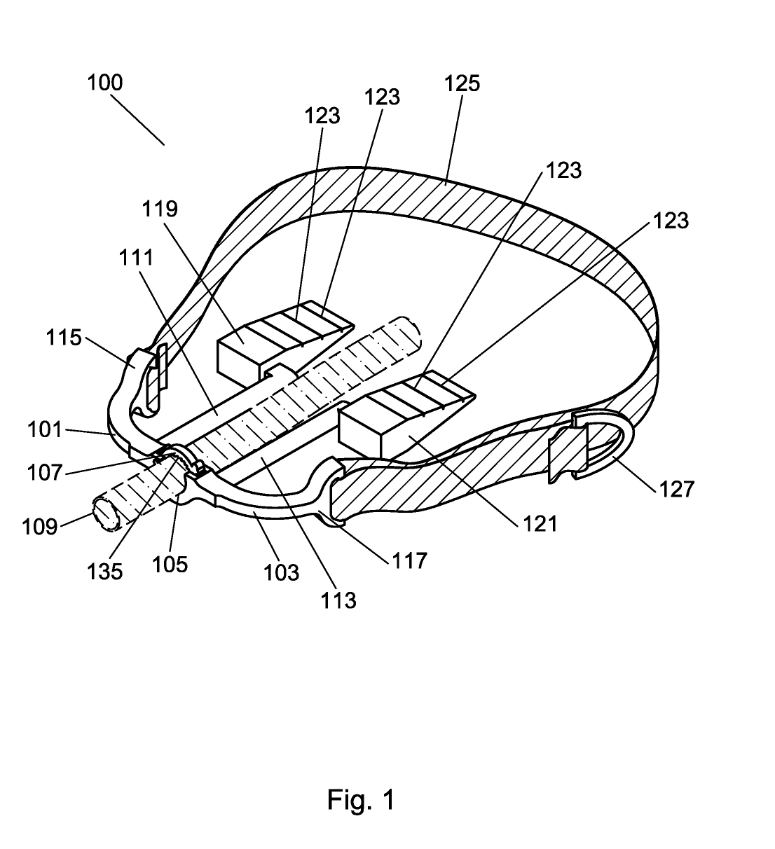

Turning first to FIG. 1, a perspective view of the Endotracheal Tube Securement Device can be seen. A first limb 101 and a second limb 103 form the primary structure of the device, and are joined or otherwise attached or manufactured together, with a tube rest 105 between each limb section. The tube rest 105 may be semi-circular or otherwise conformal to a tube 109 such as an endotracheal tube. A tube retainer 107 can also be seen above the tube rest 105, and may be hinged or otherwise movably attached to the tube rest 105 or a section of the first limb 101 or the second limb 103. The hinge, evident where the tube retainer 107 is fixed to the tube rest 105, may be a plastic or metal pin, or may be a live hinge integral with surrounding plastic of the device. The placement of the hinge may vary, as may the way in which the tube rest 105 and the tube retainer 107 cooperatively interact to ultimately retain and secure a tube such as the tube 109 depicted in the drawings. The tube retainer 107 may also have a catch, retention device, a quick and easy release, or similar securement means to retain and secure a tube 109 in the tube rest 105 without undesired movement in any direction (longitudinal, peripheral, axial, rotational, latitudinal, etc.). The tube retainer 107 may be of various geometries and styles, with many variations possible to assure a firm grip in immobilizing the endotracheal tube. The tube retainer 107 may also have retaining teeth 135 along the inner surface that makes contact with the endotracheal tube 109 in order to further secure the endotracheal tube 109. The retaining teeth may be grooves, notches, or other protrusions that push into the endotracheal tube 109 for further retention thereof.

Each limb of the device is curved or otherwise shaped to generally conform to a patient's face. The curve of each limb will generally be concave along the side closest to a patient's face so as to conform to the patient's face in an unobtrusive manner without contacting the patient's face. A first strap retainer 115 is attached or integrally formed with the first limb 101, and a second strap retainer 117 is likewise attached or integrally formed with the second limb 103. The strap retainers are designed to accommodate a strap such as the strap 125 depicted in FIG. 1, and may, in some embodiments of the present invention, provide a profile larger than that of the related limb in order to receive the strap 125. Other hardware elements may also be included in each strap retainer, such as pins, struts, hook and loop fasteners, snaps, retainers, or the like. The strap 125 is made from a flexible material such as a nylon, rubber, or other textile, and may, in some embodiments of the present invention, include an adjustment means 127 such as a buckle, latch, clasp, D-ring hook and loop fastener, or the like. The adjustment means 127 allows the strap 125 to vary in length to best accommodate the physical attributes of the patient. Protruding from the first limb 101 is a first strut 111 that may originate from the inner side of the first limb 101, and may be round, square, rectangular, or of other geometries. This first strut 111 is attached to or integrated with the first limb 101 at its proximal end. The first strut 111 makes an outward turn at its distal end in a direction away from an installed tube 109, or has a fixture at its distal end for attachment or co-forming of a first bite wedge 119. In a similar manner, protruding from the second limb 103 is a second strut 113 that may originate from the inner side of the second limb 103, and may be round, square, rectangular, or of other geometries. This second strut 113 is attached to or integrated with the second limb 103 at its proximal end. The second strut 113 also makes an outward turn at its distal end, or has a fixture at its distal end for attachment or co-forming of a second bite wedge 121. In some embodiments of the present invention, the struts follow a path that is generally parallel with an installed tube 109 so as to place each bite wedge into the oral cavity of a patient. In some embodiments of the present invention, the struts are generally orthogonal with their related limbs. In some embodiments of the present invention, the struts may be reinforced with various materials, or may contain a lumen or other conduit for removal or placement of fluids from outside the oral cavity of a patient, as will be further described herein.

The endotracheal tube securement device 100, including the main components of the limbs, tube rest and retainer, and struts, may be made from a plastic such as acrylonitrile butadiene styrene (ABS), polyethylene, polypropylene, polystyrene, polyvinyl chloride, polytetrafluoroethylene, or the like. Bioplastics may also be used in some embodiments of the present invention. The device may be injection molded, machined, printed, or otherwise fabricated using known production techniques. Other materials such as stainless steel or other clinically acceptable alternatives may also be used. However, it is envisioned that the disposable or recyclable aspects of the device will be important, along with low cost.

The first bite wedge 119 and the second bite wedge 121 not only provide for a comfortable way for the endotracheal tube securement device 100 to be securely retained in the oral cavity of a patient, but also provide a comfortable rest for the jaw of a patient while maintaining the necessary opening for the tube 109. Additionally, the bite wedges prevent the patient from biting on the tube 109 and cutting off the respirator cycle when the patient regains consciousness or is restless, but clinically must still remain on assisted breathing. The bite wedges provide a natural locking in place of the securement device 100 while the patient bites down on both bite wedges, and prevents the tube 109 from being pulled out accidentally or deliberately, causing problems with subsequent intubation. The bite wedges are generally wedge shaped, as the name implies, but may have a flattened or recessed top or bottom to facilitate proper alignment with teeth and the general oral cavity. The bite wedges may also vary in geometric attributes, have rounded edges, or otherwise vary in shape for best functionality and comfort. Further, the bite wedges may be manufactured in various sizes and geometries for differing patients or clinical applications. The bite wedges are made from a soft durometer material such as a medical grade urethane or a rubber that is medically safe for oral applications. Bite wedges may further be provided in different durometers for differing clinical applications. In some embodiments of the present invention, cut lines 123 may be placed on each bite wedge to facilitate uniform trimming of a bite wedge to better fit a patient's physical jaw profile. The cut lines 123 may simply be markings with an ink or dye, or they may also be scored or otherwise cut into the bite wedge. The bite wedges may also have inserts or other geometric features to facilitate attachment of the bite wedge to the strut. Other features, such as drain ports, porous openings, channels, or the like, may also be included to facilitate good clinical hygiene.

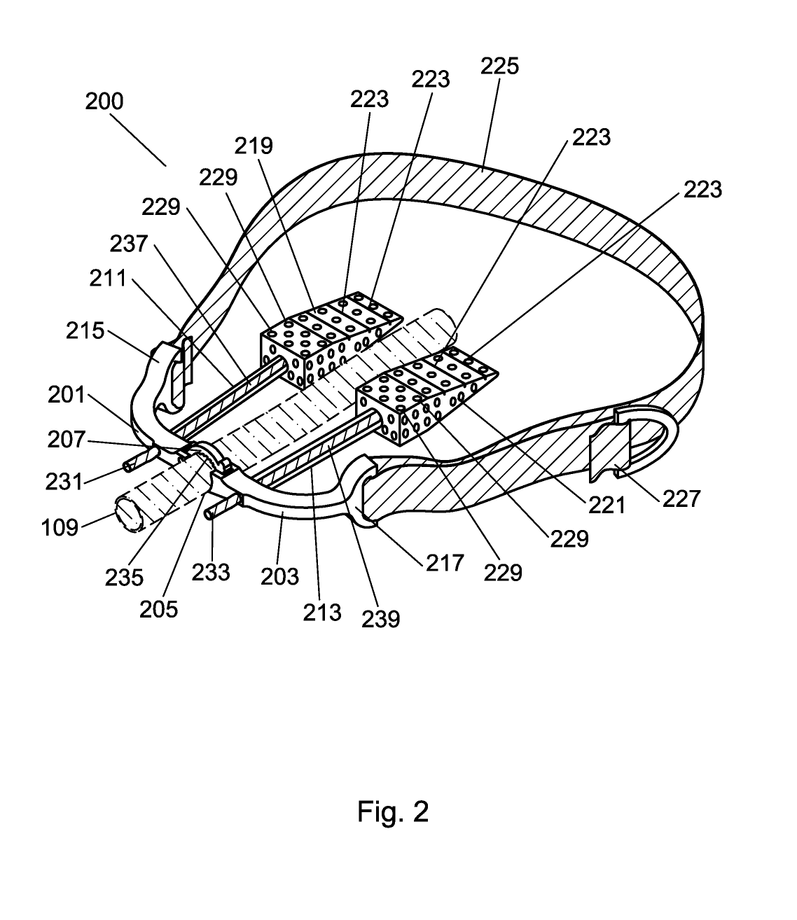

FIG. 2 is a perspective view of an alternate embodiment of the endotracheal tube securement device. This embodiment employs porous or draining bite wedges to facilitate good oral hygiene. The porous bite wedges are in fluid communication with a lumen or conduit in, adjacent to, or in proximity to each strut and connect with, or continue from, external suction ports for clinical attachment of a suction device.

A first limb 201 and a second limb 203 form the primary structure of the device, and are joined or otherwise attached or manufactured together, with a tube rest 205 between each limb section. The tube rest 205 may be semi-circular or otherwise conformal to a tube 109 such as an endotracheal tube. A tube retainer 207 can also be seen above the tube rest 205, and may be hinged or otherwise movably attached to the tube rest 205 or a section of the first limb 201 or the second limb 203. The hinge, evident where the tube retainer 207 is fixed to the tube rest 205, may be a plastic or metal pin, or may be a live hinge integral with surrounding plastic of the device. The placement of the hinge may vary, as may the way in which the tube rest 205 and the tube retainer 207 cooperatively interact to ultimately retain and secure a tube such as the tube 109 depicted in the drawings. The tube retainer 207 may also have a catch, retention device, or similar securement means to retain and secure a tube 109 in the tube rest 205 without undesired movement in any direction (longitudinal, peripheral, axial, rotational, latitudinal, etc.). The tube retainer 207 may be of various geometries and styles, with many variations possible. The tube retainer 207 may also have retaining teeth 235 along the inner surface that makes contact with the endotracheal tube 109 in order to further secure the endotracheal tube 109. The retaining teeth 235 may be grooves, notches, or other protrusions that push into the endotracheal tube 109 for further retention thereof.

Each limb of the device is curved or otherwise shaped to conform to a patient's face. The curve of each limb will generally be concave along the side closest to a patient's face so as to conform to the patient's face in an unobtrusive manner. A first strap retainer 215 is attached or integrally formed with the first limb 201, and a second strap retainer 217 is likewise attached or integrally formed with the second limb 203. The strap retainers are designed to accommodate a strap such as the strap 225 depleted in FIG. 2, and may, in some embodiments of the present invention, provide a profile larger than that of the related limb in order to receive the strap 225. Other hardware elements may also be included in each strap retainer, such as pins, struts, hook and loop fasteners, snaps, retainers, or the like. The strap 225 is made from a flexible material such as a nylon, rubber, or other textile, and may, in some embodiments of the present invention, include an adjustment means 227 such as a buckle, latch, clasp, D-ring, hook and loop fastener, or the like. The adjustment means 227 allows the strap 225 to vary in length to best accommodate the physical attributes of the patient. Protruding from the first limb 201 is a first strut 211 that may originate from the inner side of the first limb 201, and may be round, square, rectangular, or of other geometries. This first strut 211 is attached to the first limb 201 at its proximal end. The first strut 211 makes an outward turn in a direction away from an installed tube 109, or has a fixture at its distal end for attachment of a first bite wedge 219. In a similar manner, protruding from the second limb 203 is a second strut 213 that may originate from the inner side of the second limb 203, and may be round, square, rectangular, or of other geometries. This second strut 213 is attached to the second limb 203 at its proximal end. The second strut 213 also makes an outward turn, or has a fixture at its distal end for attachment of a second bite wedge 221. In some embodiments of the present invention, the struts follow a path that is generally parallel with an installed tube 109 so as to place each bite wedge into the oral cavity of a patient so that they may be anchored by a patient's teeth. In some embodiments of the present invention, the struts are generally orthogonal with their related limbs. The struts may also enter into the bite wedges without a turn, such as directly into a face of each bite wedge, as depicted in FIG. 2 for example. In some embodiments of the present invention, the struts may be reinforced with various materials, or may contain a lumen or other conduit for removal or placement of fluids from outside the oral cavity of a patient. For example, as depicted in FIG. 2, the bite wedges are porous or may contain porous openings such as the openings 229. These openings 229 may vary in diameter, and may be complimentary to porous material used for the bite wedges. The bite wedges may have openings 229 throughout their surfaces. The openings 229 may vary in size, but serve to provide a porous path through which oral fluids may be absorbed and drain into the suction conduit of the related strut and suction port. Further, the openings 229 may be a single opening, port, slit, or other such structure to facilitate suctioning, drainage, and proper hygiene for the patient. The opening or openings may be in proximity to the bite wedges or placed there through, and be in fluid communication with suction conduits such as tubes or the like. The suction conduit of each strut may be suction tubing such as the first suction tube 237 contained within an open channel in the first strut 211. The suction tube 237 is in fluid communication with the first bite wedge 219 and the first suction port 231. In some embodiments of the present invention, the first suction port 231 comprises the suction tube 237. In a similar way, a second suction tube 239 is contained within an open channel in the second strut 213. The suction tube 239 is in fluid communication with the second bite wedge 221 and the second suction port 233. One or both bite wedges may be structured to provide suction either through the bite wedge or in proximity thereof. In some embodiments of the present invention, the second suction port 233 comprises the suction tube 239. The bite wedges may have an opening or channel such as the suction fitting 801 depicted in FIG. 8. This suction fitting is in fluid communication with the porous openings 229, and connect with each strut. The first strut 211 and the second strut 213 may also be hollow, or contain or be coupled with a lumen or conduit to allow fluid to be aspirated. In some embodiments, a suction tube or similar conduit could be inserted through the hollow strut. The lumen of each strut connects with the suction fitting on the related bite wedge. A first suction port 231 can be seen connected to the first strut 211 and is a generally hollow structure that is in fluid communication with the lumen or conduit of the first strut 211, which is in turn in fluid communication with the porous openings 229 of the first bite wedge 219. In a similar manner, a second suction port 233 can be seen connected to the second strut 213 and is a generally hollow structure is in fluid communication with the lumen or conduit of the second strut 213, which is in turn in fluid communication with the porous openings 229 of the second bite wedge 221. The suction ports may vary in size, and may have additional fixturing to accommodate various types of suction or aspiration equipment to be attached thereto. The suction ports may be molded with or integral to the limbs, or may be attached to the limbs in a secondary operation. The suction ports may also be the end of a tube that is fitted inside the lumen of each strut. Various other production techniques may be employed to create the suction conduit path from each suction port through the relevant bite wedge.

The endotracheal tube securement device 200, including the main components of the limbs, tube rest and retainer, struts and suction ports, may be made from a plastic such as acrylonitrile butadiene styrene (ABB), polyethylene, polypropylene, polystyrene, polyvinyl chloride, polytetrafluoroethylene, or the like. Bioplastics may also be used in some embodiments of the present invention. The device may be injection molded, machined, printed, or otherwise fabricated using known production techniques. Other materials such as stainless steel or other clinically acceptable alternatives may also be used. However, it is envisioned that the disposable or recyclable aspects of the device will be important, along with low cost.

The first bite wedge 219 and the second bite wedge 221 not only provide for a comfortable way for the endotracheal tube securement device 200 to be securely retained in the oral cavity of a patient, but also provide a comfortable rest for the jaw of a patient while maintaining the necessary opening for the tube 109. Additionally, the bite wedges prevent the patient from biting on the tube 109 and cutting off the respirator cycle when the patient regains consciousness or is restless, but clinically must still remain on assisted breathing. The bite wedges provide a natural locking in place of the securement device 200 while the patient bites down on both bite wedges, and prevents the tube 109 from being pulled out accidentally or deliberately, causing problems with subsequent intubation. The bite wedges are generally wedge shaped, as the name implies, but may have a flattened or recessed top or bottom to facilitate proper alignment with teeth and the general oral cavity. Further, the bite wedges may be manufactured in various sizes and geometries for differing patients or clinical applications. The bite wedges are made from a soft durometer material such as a medical grade urethane or a rubber that is medically safe for oral applications. Bite wedges may further be provided in different durometers for differing clinical applications, and may be co-molded or separately molded. In some embodiments of the present invention, cut lines 223 may be placed on each bite wedge to facilitate uniform trimming of a bite wedge to better fit a patient's physical profile. The cut lines 223 may simply be markings with an ink or dye, or they may also be scored, or otherwise cut into the bite wedge. The bite wedges may also have inserts or other geometric features to facilitate attachment of the bite wedge to the strut. Other features, such as drain ports, porous openings, channels, or the like, may also be included to facilitate good clinical hygiene. The bite wedges may also vary in geometric attributes, have rounded edges, or otherwise vary in shape for best functionality and comfort.

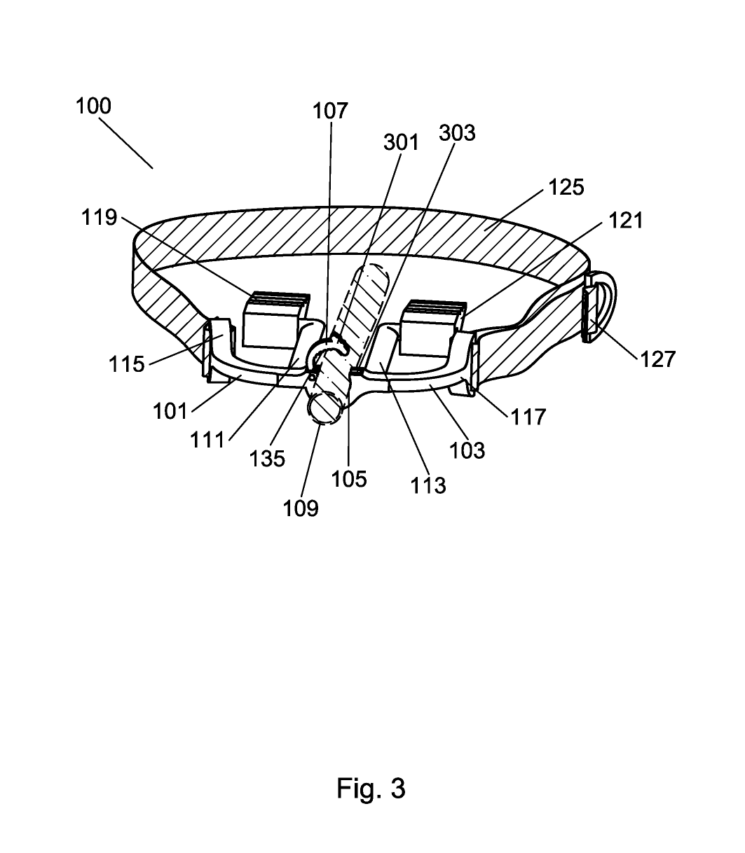

FIGS. 3-8 depict various views of the endotracheal tube securement device of FIG. 1. While the embodiment depicted is that shown in FIG. 1, the general attributes depicted in FIGS. 3-8 are equally applicable to the embodiment depicted in FIG. 2.

FIG. 3 is a front perspective view of the endotracheal tube securement device of FIG. 1. In this view, the tube retainer 107 is in the open, or unsecured, position. A retainer lock 301 can be seen at the free end of the tube retainer 107. The retainer lock 301 may be shaped with a bevel. Here, or a series of progressive bumps or retaining elements to engage with a retainer lock receiver 303. The retainer lock receiver 303 is an open space that may also contain a ledge, notch, or other feature to engage with the retainer lock 301. Other embodiments of the present invention may employ differing tube retainer arrangements, and variation of the tube retainer itself does not depart from the spirit and broad scope of the present invention as described and envisioned herein.

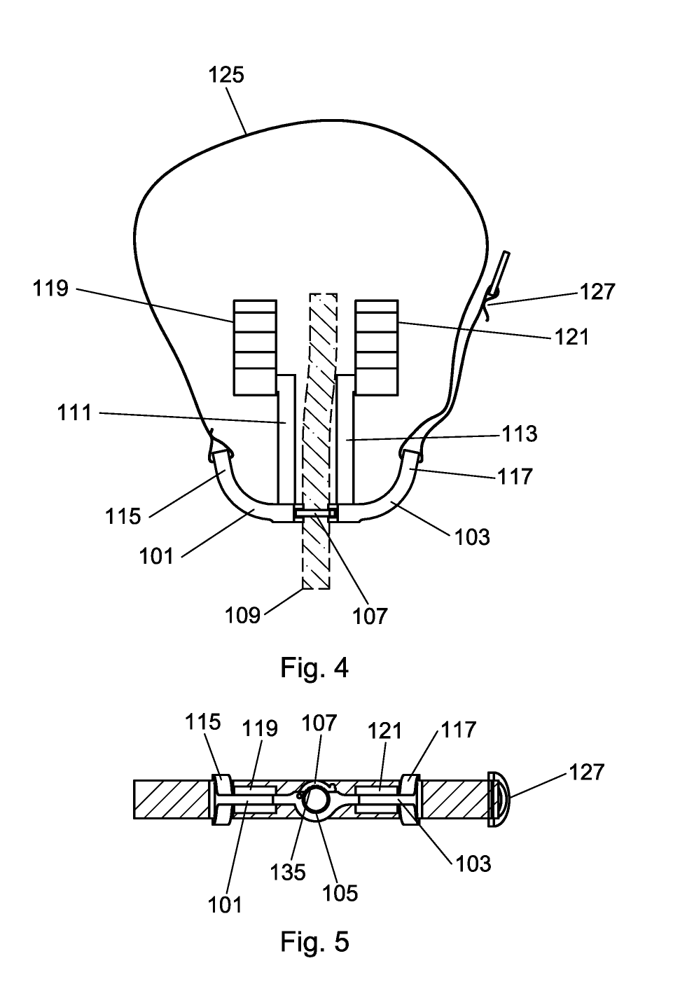

FIG. 4 is a top plan view of the endotracheal tube securement device of FIG. 1 showing the tube 109 in cross-hatching and secured in place.

FIG. 5 is a front plan view of the endotracheal tube securement device of FIG. 1 showing the tube retainer 107 closed securely around the tube 109.

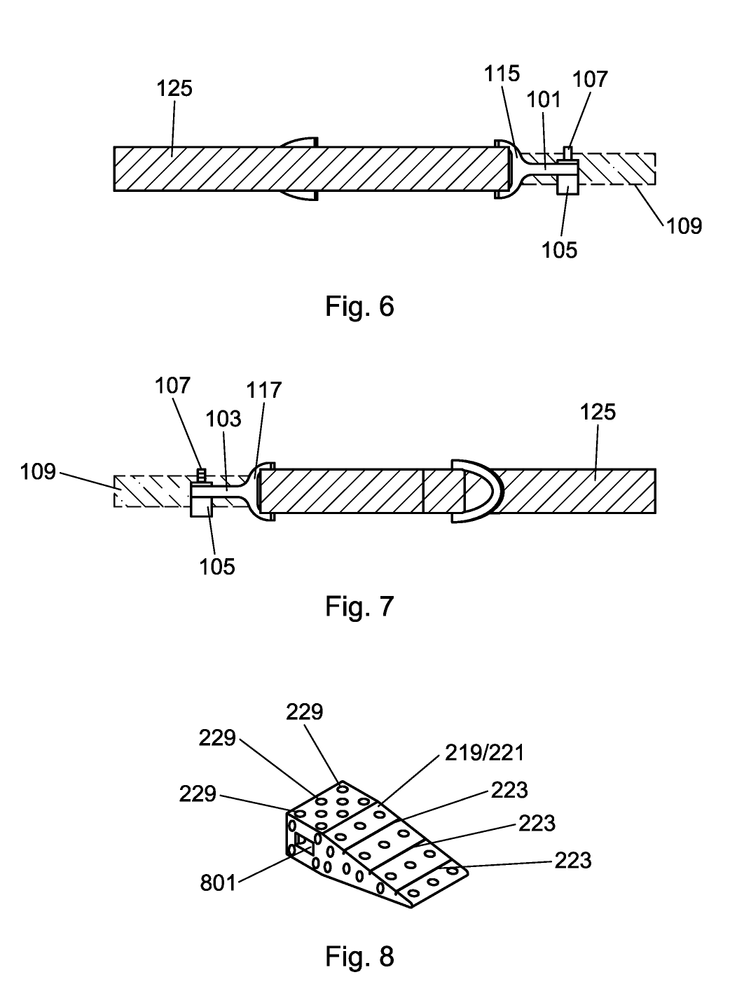

FIG. 6 is a first side plan view of the endotracheal tube securement device of FIG. 1 and FIG. 7 is a second side plan view of the endotracheal tube securement device of FIG. 1.

FIG. 8 is a perspective view of a bite wedge 219 or 221 of the present invention. As previously described, the bite wedge is porous, and may comprise porous openings 229 throughout, or in specific locations. The cut lines 223 can also be seen along with a bite wedge suction fitting 801. The suction fitting 801 shown in FIG. 8 is a rectangular opening. Such an opening may also be round, oval, square, or other geometry. The suction fitting 801 is in fluid communication with porous openings 229 to facilitate drainage and suction of oral fluids. The suction fitting 801 may simply be a specific size opening, or may include other fixtures such as flanges, couplings, barbs, or the like. The suction fitting 801 is attached to the distal end of each strut or the suction tube within each strut, and may be pressure fit, heat fit, glued, ultrasonically welded, molded, or the like.

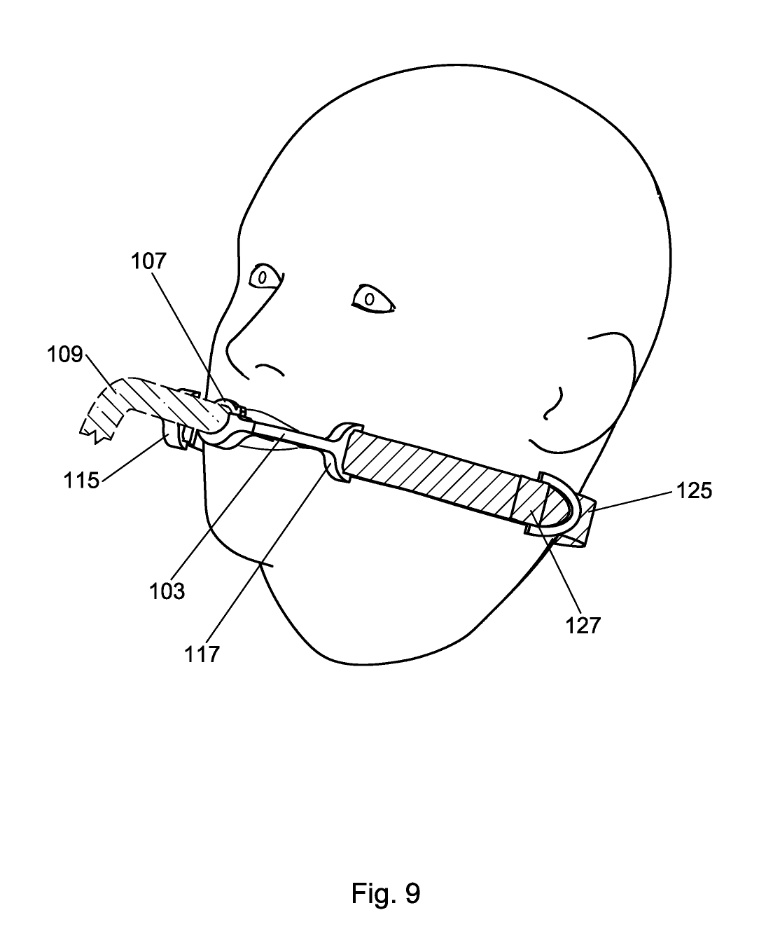

Lastly, FIG. 9 depicts the endotracheal tube securement device in use. The limbs can be seen conforming with, but not touching, the patient's face. The strap 125 secures the device around the patient's head, and the endotracheal tube 109 is securely retained by the tube retainer 107.

To use the endotracheal tube securement device, the device is temporarily fitted to the patient. The strap is adjusted and the bite wedges are trimmed to an appropriate size as necessary. The device is then removed, and the endotracheal tube is fitted to the patient, a process commonly known as intubation. Once intubation is complete, the endotracheal tube securement device 100 (or 200) is placed in the oral cavity by way of the bite wedges and struts, the strap is placed around the patient's head, and the tube retainer is closed and secured. Adequate fit is checked and adjustments made as necessary. The endotracheal tube securement device facilitates not only good suctioning and oral hygiene for the patient, but also allows for saline irrigation by use of the structural features of the device.

It is, therefore, apparent that there has been provided, in accordance with the various objects of the present invention, an endotracheal tube securement device.

While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of this specification, claims and drawings appended herein.

What is claimed is:

1. An endotracheal tube securement device comprising:
a first limb that generally conforms to a patient's face and having a first strap retainer;
a second limb that generally conforms to a patient's face and having a second strap retainer;
the first limb joined to the second limb at a junction forming a tube rest for a tube;
a tube retainer movably attached to the tube rest;
a first stmt protruding from the first limb, the first strut retaining a first bite wedge; and
a second strut protruding from the second limb, the second strut retaining a second bite wedge.

2. The endotracheal tube securement device of claim 1, further comprising a strap for coupling with the first strap retainer and the second strap retainer.

3. The endotracheal tube securement device of claim 1, further comprising an endotracheal tube.

4. The endotracheal tube securement device of claim 1, further comprising cut lines for adjustment of the first bite wedge and the second bite wedge.

5. The endotracheal tube securement device of claim 1, further comprising retaining teeth on the tube retainer.

6. The endotracheal tube securement device of claim 1, further comprising a hinge moveably connecting the tube retainer to the tube rest.

7. The endotracheal tube securement device of claim 1, wherein the tube is an endotracheal tube.

8. An endotracheal tube securement device comprising:
a first limb that generally conforms to a patient's face and having a first strap retainer;
a second limb that generally conforms to a patient's face and having a second strap retainer;
the first limb joined to the second limb at a junction forming a tube rest for a tube;
a tube retainer movably attached to the tube rest;
a first suction port;
a first strut protruding from the first limb, the first strut retaining a first bite wedge having porous openings; and
a second strut protruding from the second limb, the second strut retaining a second bite wedge having porous openings.

9. The endotracheal tube securement device of claim 8, further comprising a first suction tube in fluid communication with the first suction port.

10. The endotracheal tube securement device of claim 8, further comprising a second suction port in fluid communication with a patient's oral cavity.

11. The endotracheal tube securement device of claim 10, further comprising a second suction tube in fluid communication with the second suction port and a first suction tube in fluid communication with the first suction port.

12. The endotracheal tube securement device of claim 9, wherein the first suction tube comprises a generally hollow first strut.

13. The endotracheal tube securement device of claim 11, wherein the first suction tube comprises a generally hollow first strut and the second suction tube comprises a generally hollow second strut.

14. The endotracheal tube securement device of claim 11, wherein the porous openings of the first bite wedge are in fluid communication with the first suction tube and the porous openings of the second bite wedge are in fluid communication with the second suction tube.

15. The endotracheal tube securement device of claim 8, wherein the first bite wedge and the second bite wedge each comprise a suction fitting.

16. The endotracheal tube securement device of claim 8, further comprising a strap for coupling with the first strap retainer and the second strap retainer.

17. The endotracheal tube securement device of claim 8, further comprising an endotracheal tube.

18. The endotracheal tube securement device of claim 8, further comprising cut lines for adjustment of the first bite wedge and the second bite wedge.

19. The endotracheal tube securement device of claim further comprising retaining teeth on the tube retainer.

20. The endotracheal tube securement device of claim 8, further comprising a hinge moveably connecting the tube retainer to the tube rest.
